(19) United States
(12) Patent Application Publication
(10) Pub. No.: US 2024/0341901 A1
(43) Pub. Date: Oct. 17, 2024

(54) PROJECTION FOR INTERVENTIONAL MEDICAL PROCEDURES

(71) Applicant: Hologic, Inc., Marlborough, MA (US)

(72) Inventors: Adrian HUNSDON, Southbury, CT (US); David WOLFF, Hockessin, DE (US); Shawn HOCHSTETLER, Sandy Hook, CT (US)

(73) Assignee: Hologic, Inc., Marlborough, MA (US)

(21) Appl. No.: 18/580,325

(22) PCT Filed: Jul. 25, 2022
(86) PCT No.: PCT/US2022/038181

§ 371 Date: Jan. 18, 2024

Related U.S. Application Data
(60) Provisional application No. 63/226,003, filed on Jul. 27, 2021.

(51) Int. Cl. A61B 90/35; A61B 90/00

(52) U.S. Cl. CPC A61B 90/35; A61B 2090/366

(57) ABSTRACT

Systems and methods for projecting a visual indicator onto a breast are disclosed. To better estimate an incision location or a location to apply an anesthetic, a visual indicator may be shown on the breast to indicate an intersection point of an interventional element and a surface of the breast. The visual indicator may be projected from a light source. The source may be positioned on a guidance system for an element near the element itself. The visual indicator may be moved based on a distance between the light and the surface of the breast (e.g., to counteract a parallax effect).

### CROSS-REFERENCE TO RELATED APPLICATIONS

This application is being filed on Jul. 25, 2022, as a PCT Patent International Application and claims the benefit of priority to U.S. Provisional Application No. 63/226,003, filed Jul. 27, 2021, which application is hereby incorporated in its entirety by reference.

### INTRODUCTION

Medical imaging is used for detection of cancerous cells in breast tissue. A plurality of different imaging processes, image acquisition parameters, and image processing techniques are used to enhance images for better detection of abnormal tissue.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

Examples of the present disclosure describe systems and methods relating to projecting a visual indicator onto a surface of a breast.

In one aspect, the technology relates to a method for projecting an incision indicator onto a breast of a patient, the method including: compressing the breast; acquiring, while compressing the breast, an x-ray image of the breast; receiving an indication identifying at least one target region in the breast, based at least on the x-ray image; detecting that an arm securing an interventional element is positioned such that a path of the interventional element intersects with the breast at the at least one target region and intersects at a surface of the breast at a target incision point; and projecting an incision indicator onto the surface of the breast at the target incision point while illuminating the at least a portion of the breast with task lighting. In an example, the method includes adjusting the projected incision indicator based on a change in a distance between the surface of the breast and a projector projecting the incision indicator. In another example, the method further includes adjusting the projected incision indicator to counteract a parallax effect. In yet another example, at least one target region includes a first target region and a second target region, the method further including: receiving a selection of the first target region, wherein the incision indicator is projected based on the path of the interventional element intersecting the first target region; receiving a selection of the second target region; detecting that the arm is repositioned such that the path of the interventional element intersects with the second target region; and adjusting the projection of the incision indicator to intersect the path of the interventional element at the second target region on the surface of the breast.

In an example of the above aspect, projecting the incision indicator is automatic based on detecting that the arm is positioned such that the path of the insertion element intersects with the breast at the at least one target region. In another example, the method further includes receiving an indication to review projection of the incision indicator; and terminating projection of the incision indicator. In yet another example, the task lighting is illuminated subsequent to terminating projection of the incision indicator. In still another example, the incision indicator is at least one of: a crosshair; a dot; an oval; a rectangle; and a line. In still another example, the incision indicator includes a selectable color.

In another aspect, the technology relates to an apparatus for projecting an incision indicator onto a breast of a patient, the apparatus including: an x-ray source capable of selectively moving relative to the breast; an x-ray detector; a compression system for compressing the breast, the compression system disposed between the x-ray source and the x-ray detector; an arm for securing an interventional element; a task lighting source disposed between the x-ray source and the compression system; an indicator source disposed between the x-ray source and the compression system, wherein the indicator source is coupled to the arm; a processor; and memory storing instructions that, when executed by the processor, cause the apparatus to perform a set of operations including: illuminating at least a portion of the breast with the task lighting source; detecting that the arm is in a position such that a path of the interventional element intersects with a surface of the breast at a target incision point; and projecting an incision indicator from the indicator source onto the surface of the breast at the target incision point. In an example, the tasking lighting source and the indicator source are the same. In another example, the tasking lighting source and the indicator source are a projector. In yet another example, the set of operations further includes: determining a location of the incision indicator in a projection image of the projector, based on a distance between the projector and the surface of the breast. In still another example, the distance between the projector and the surface of the breast is based on the position of the arm.

In an example of the above aspect, the indicator source is calibrated based on a calibration position of the arm. In another example, the indicator source is a micro electrical mechanical system (MEMS) including a mirror that is adjustable to control reflection of a laser beam to project the incision indicator onto the surface of a breast. In yet another example, the mirror of the MEMS is adjusted based on a distance between the indicator source and the surface of the breast. In still another example, the indicator source is positioned at an end of the arm. In still another example, the interventional element is a needle or a wire.

In another aspect, the technology relates to an apparatus for projecting an incision indicator onto a breast of a patient, the apparatus including: an x-ray source capable of selectively moving relative to the breast; an x-ray detector; a compression system for compressing the breast, the compression system disposed between the x-ray source and the x-ray detector; an arm for securing an interventional element, the arm disposed between the x-ray source and the compression system; and a projector coupled to an end of the arm, the projector capable of selectively projecting task lighting and an incision indicator onto a surface of the breast based at least on a position of the arm and a target incision point.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

The accompanying figures illustrate one or more aspects of the disclosed methods and systems. In the appended figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label. Non-limiting and non-exhaustive examples are described with reference to the following figures:

FIG. 1A depicts a schematic view of an example imaging system.

FIG. 1B depicts a perspective view of the imaging system of FIG. 1A.

FIG. 1C depicts an assembly mountable on the imaging system of FIG. 1A.

FIG. 1D depicts the assembly of FIG. 1C mounted on the imaging system of FIG. 1A.

FIG. 1E depicts a perspective view of the light source of the imaging system of FIG. 1A.

FIG. 2 depicts a prior art configuration of an end of an angular support arm of an imaging system.

FIG. 3 depicts an imaging system with two light sources, instead of a single light source.

FIGS. 4A-4B depict top-down views of a breast compressed by a compression device illuminated by task lighting and a visual indicator.

FIG. 5 depicts an example method for projection onto a breast of a patient.

FIG. 6 illustrates an example suitable operating environment for a projection system.

While examples of the disclosure are amenable to various modifications and alternate forms, specific examples have been shown by way of example in the drawings and are described in detail below. The intention is not to limit the scope of the disclosure to the particular examples described. On the contrary, the disclosure is intended to cover all modifications, equivalents, and alternatives falling within the scope of the disclosure and the appended claims.

### DETAILED DESCRIPTION

Various aspects of the disclosure are described more fully below, with reference to the accompanying drawings, which show specific example aspects. However, different aspects of the disclosure may be implemented in many different forms and should not be construed as limited to the aspects described herein; rather, these aspects are provided so that this disclosure will be thorough and complete and will fully convey the scope of the aspects to those skilled in the art. Aspects may be practiced as methods, systems, or devices. The following detailed description is, therefore, not to be interpreted in a limiting sense.

Breast cancer is one of the leading causes of cancer-related mortality of women. Abnormalities may be identified in the breast tissue by implementing one or more imaging techniques such as breast CT, breast tomosynthesis, and digital mammography. If an abnormality is identified in breast tissue, confirmatory localization, biopsy, or other procedures may be performed. The technologies described herein include image-guided systems and methods for performing various interventional medical procedures with a wide range of implements. Such implements may include, but are not limited to, localization elements such as a localization needle, wire, or implant; a radiolucent seed; a biopsy needle; or any other element that is insertable into the breast for identification, marking, biopsy, and/or removal of identified, potentially abnormal tissue. A common application, described in more detail herein, includes subcutaneous insertion of a localization element into the breast to aid in future assessment of the potentially abnormal tissue. Another common application includes insertion of a biopsy needle to remove discrete portions of a suspected abnormality. While the terms “localization” and “biopsy” are used primarily herein for illustrative purposes, the present disclosure is not limited to those particular procedures; rather, any interventional procedures that may benefit from image-guided implement positioning are contemplated.

Prior to inserting a localization element into a breast, a small incision or nick may be made at the surface in the skin of the breast where the localization element is to be inserted. The small incision is often made by a doctor with a scalpel. The incision may reduce movement of breast tissue during subcutaneous insertion of the localization element through the incision. Additionally, the breast tissue is often anesthetized to enhance patient comfort. The localization element may be positioned in an x-direction and a y-direction by a guidance system and manually moved in a z-direction (toward or away from the breast) by a doctor.

Traditionally, to determine where to make the incision, a guided localization element may be moved in the z-direction towards the breast until an intersection point with the skin of the breast may be visually estimated. The incision is then made at the visually-estimated intersection point of the localization element at the skin or surface of the breast. In an example, the localization element may be advanced until intersecting with the tissue, then backed off so that an incision may be made based on a visual estimation of the intersection point. This approach, however, requires visual estimation for an incision and wasted time moving a localization element for the estimation.

To better estimate an incision location and/or a location to apply an anesthetic, a visual indicator may be projected onto the surface of the breast to indicate an intersection point of the localization element and the breast. The visual indicator may be projected from a light source. The light source may be positioned on a guidance system near the localization element. If the light source is positioned to project a visual indicator concentrically with, and parallel to, the localization element, then no adjustment of the light source or visual indicator would be needed based on the distance of the localization element from the breast. Examples of concentric and parallel placement of the source include temporarily replacing the localization element with the light source, placing the light source on the localization element, or placement of the light source concentrically and parallel with the localization element. Each of these concentric and parallel placements, however, have shortcomings. Temporarily mounting a light source in place of a localization element requires time to remove and replace the localization element and may result in accidental movement of the guidance system and/or improper securing of the localization element. Placing a light source on a localization element may result in the light source being inserted into the breast with the localization element, which could result in dislodgement of the light source inside of the breast, greater patient discomfort (e.g., a bigger object being inserted into the breast), sanitation concerns, and lack of power routed to the source. Moreover, a light source placed concentric and parallel with the localization element would either prevent intersection of the localization element and the breast (e.g., if placed between the localization element and the breast) or the visual indicator's path would be blocked by the localization element (e.g., if the localization element is placed between the source and the breast). Thus, concentric and parallel placement of a light source may not be practical or desirable.

Alternatively, the light source and localization element may be eccentric. If the source and localization element are eccentric, projection of the visual indicator may be subjected to a parallax effect, depending on the distance between the light source and the breast onto which the visual indicator is projected. Thus, the projection path of the visual indicator, or position of the visual indicator in a projection area, may be required or desired to be adjustable to counteract a parallax effect.

Accordingly, the present disclosure provides systems and methods for projecting a visual indicator onto a breast that is adjustable to counteract a parallax effect. In an example, the present technology provides a light source to project a visual indicator onto a breast. The light source may provide task lighting in addition to, or as an alternative to, the visual indicator. The light source may move the visual indicator within a projection area based on a distance between the light source and the breast. The distance between the light source and the breast may be detected by the light source or may be provided as an input to the light source from a guidance system positioning a localization element. The light source may be coupled to the guidance system and may share power with the guidance system.

In another example, the visual indicator may be projected onto a breast with a beam of light (e.g., a laser). The angle and/or position of the light source projecting the beam of light may be adjusted based on the distance between the light source and the breast. For example, an angle of the light source may be mechanically adjusted. Additionally or alternatively, a position of the light source relative to the localization element may be mechanically adjusted.

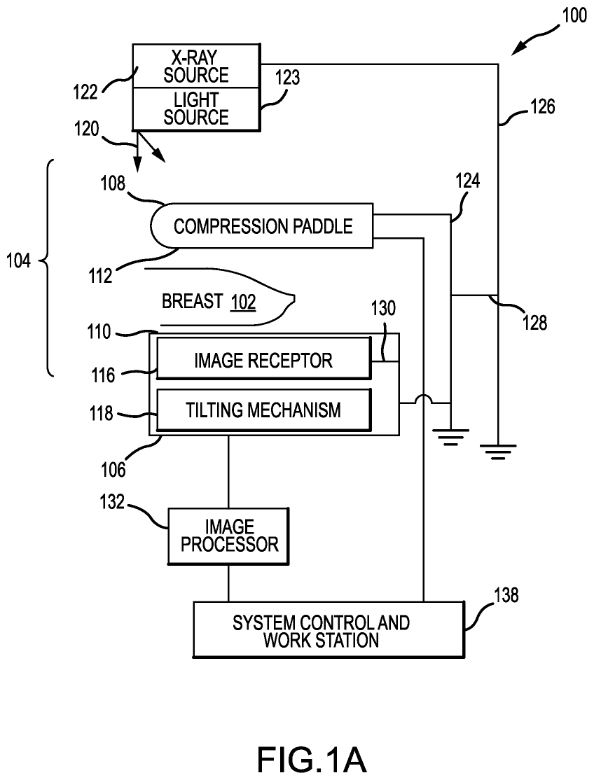

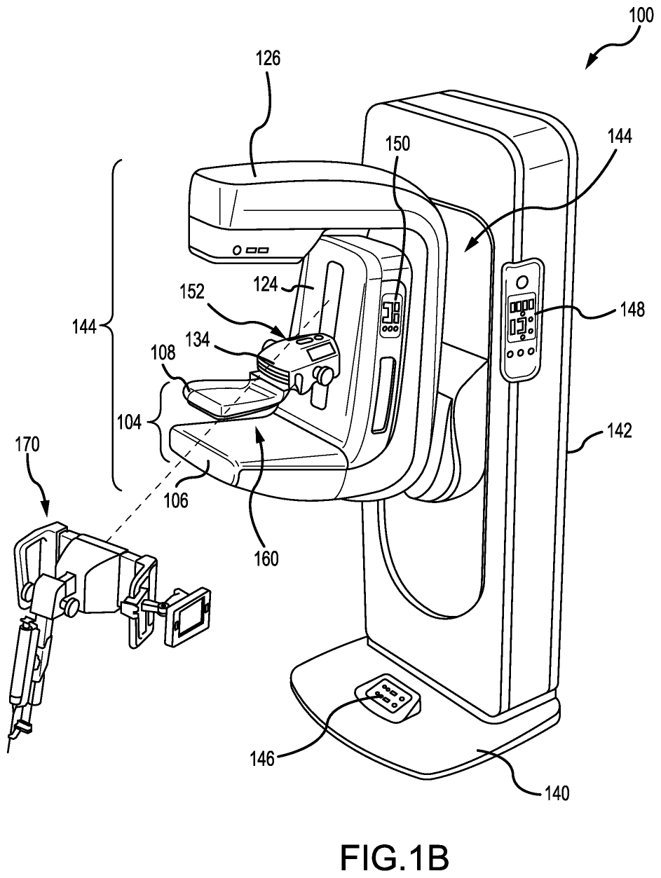

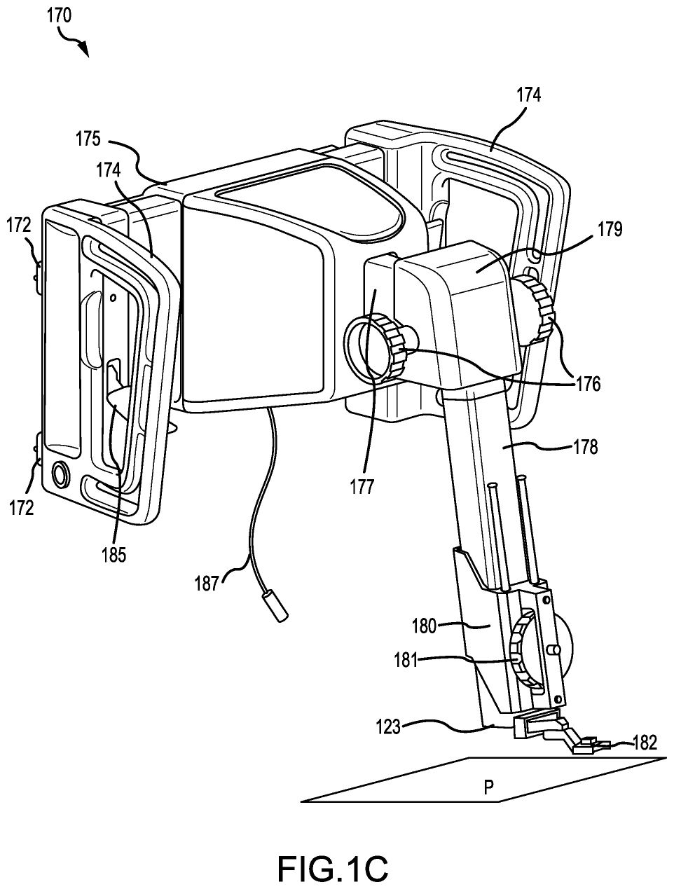

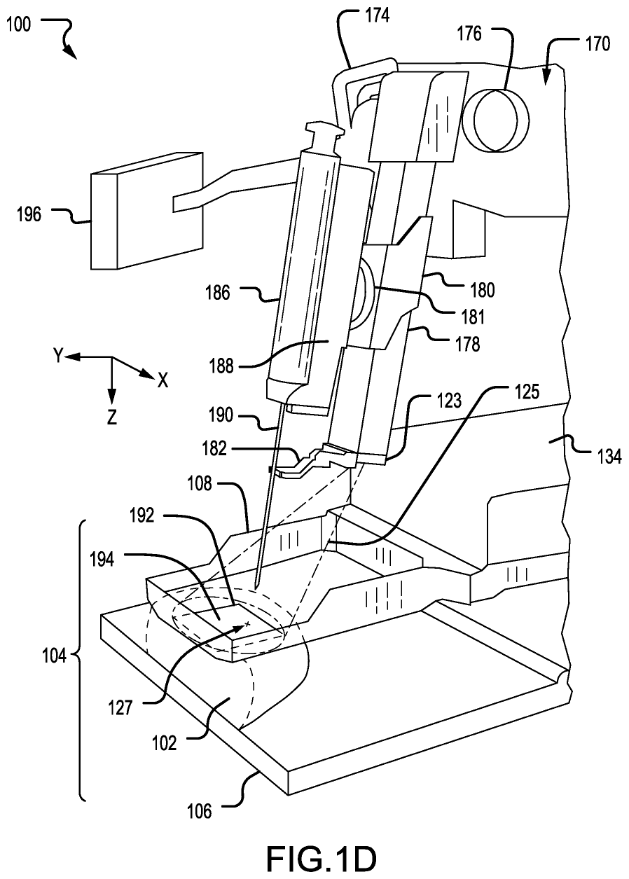

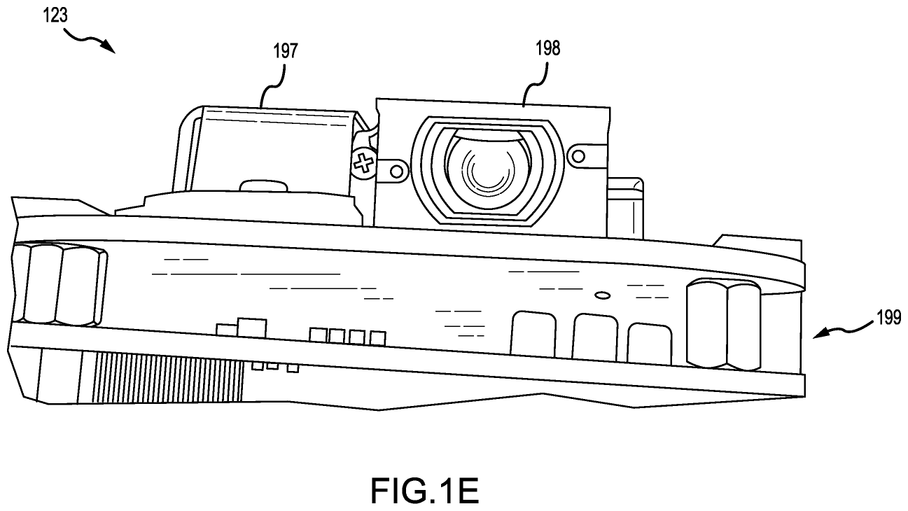

FIGS. 1A-1D show different views of an example imaging system 100 and a localization assembly 170 couplable to the imaging system. Specifically, FIG. 1A is a schematic view of the example imaging system 100. FIG. 1B is a perspective view of the imaging system 100. Referring concurrently to FIGS. 1A and 1B, not every element described below is depicted in both figures. The descriptions provided herein may be applied to either an upright (shown) or prone (not shown) imaging system 100. For simplicity, the following discussion includes examples for use with an upright breast tomosynthesis imaging system (such as the Selenia® Dimensions® breast tomosynthesis imaging system provided by Hologic, Inc.).

The imaging system 100 immobilizes a patient's breast 102 for x-ray imaging (either or both of mammography and tomosynthesis) via a breast compression immobilizer unit 104 that includes a static breast support platform 106 and a compressive element 108 in the form of a paddle. Different paddles, each having different purposes, are known in the art. Certain examples paddles are also described herein for context. The breast support platform 106 and the compressive element 108 each have a compression surface 110 and 112, respectively, that move towards each other to compress, immobilize, stabilize, or otherwise hold and secure the breast 102 during imaging procedures. In known systems, the compression surface 110, 112 is exposed so as to directly contact the breast 102. Compression surface 110 may be a rigid plastic, a flexible plastic, a resilient foam, a mesh or screen, and so on. The platform 106 also houses an image receptor 116 and, optionally, a tilting mechanism 118, and optionally an anti-scatter grid (not depicted, but disposed above the image receptor 116). The immobilizer unit 104 (otherwise referred to herein as the compression system 104) is in a path of an imaging beam 120 emanating from x-ray source 122, such that the imaging beam 120 impinges on the image receptor 116.

The immobilizer unit 104 is supported on a first support arm 124 via a compression arm 134, which is configured to be raised and lowered along the support arm 124. The x-ray source 122 is supported on a second support arm, also referred to as a tube head 126. For mammography, support arms 124, 126 can rotate as a unit about an axis 128 between different imaging orientations such as craniocaudal (CC) and mediolateral oblique (MLO), so that the imaging system 100 can take a mammogram projection image at each orientation. (The terms front, lower, and upper pertain to using a CC imaging orientation, with the patient facing the front of the imaging system, although it should be understood that other imaging orientations, including MLO, are used with the same equipment.) In operation, the image receptor 116 remains in place relative to the platform 106 while an image is taken. The immobilizer unit 104 releases the breast 102 for movement of arms 124, 126 to a different imaging orientation. For tomosynthesis, the support arm 124 stays in place, with the breast 102 immobilized and remaining in place, while at least the second support arm 126 rotates the x-ray source 122 relative to the immobilizer unit 104 and the compressed breast 102 about the axis 128. The imaging system 100 takes plural tomosynthesis projection images of the breast 102 at respective angles of the imaging beam 120 relative to the breast 102.

Concurrently and optionally, the image receptor 116 may be tilted relative to the breast support platform 106 and in sync with the rotation of the second support arm 126. The tilting can be through the same angle as the rotation of the x-ray source 122 but may also be through a different angle selected such that the imaging beam 120 remains substantially in the same position on the image receptor 116 for each of the plural images. The tilting can be about an axis 130, which can but need not be in the image plane of the image receptor 116. The tilting mechanism 118 that is coupled to the image receptor 116 can drive the image receptor 116 in a tilting motion. For tomosynthesis imaging and/or CT imaging, the breast support platform 106 can be horizontal or can be at an angle to the horizontal, e.g., at an orientation similar to that for conventional MLO imaging in mammography. The imaging system 100 can be solely a mammography system, a CT system, or solely a tomosynthesis system, or a “combo” system that can perform multiple forms of imaging. An example of such a combo system has been offered by the assignee hereof under the trade name Selenia Dimensions.

When the system is operated, the image receptor 116 produces imaging information in response to illumination by the imaging beam 120 and supplies it to an image processor 132 for processing and generating breast x-ray images. A system control and workstation unit 138 including software controls the operation of the system and interacts with the operator to receive commands and deliver information including processed-ray images.

The imaging system 100 includes a floor mount or base 140 for supporting the imaging system 100 on a floor. A gantry 142 extends upwards from the base 140 and rotatably supports both the tube head 208 and a support arm 210. The tube head 126 and support arm 124 are configured to rotate discretely from each other and may also be raised and lowered along a face 144 of the gantry 142 so as to accommodate patients of different heights. The x-ray source 122 is disposed within the tube head 208. Together, the tube head 126 and support arm 124 may be referred to as a C-arm 124.

A number of interfaces and display screens are disposed on the imaging system 100. These include a foot display screen 146, a gantry interface 148, a support arm interface 150, and a compression arm interface 152. In general, the various interfaces 148, 150, and 152 may include one or more tactile buttons, knobs, switches, as well as one or more display screens, including capacitive touch screens with graphic user interfaces (GUIs) so as to enable user interaction with and control of the imaging system 100. In general, the foot display screen 146 is primarily a display screen, though a capacitive touch screen might be utilized if required or desired.

A assembly 170, shown in greater detail in FIG. 1C and FIG. 1D, may be mountable in between the x-ray source 122 and the x-ray detector 116 (otherwise referred to herein as the image receptor 116) of the imaging system 100. The assembly 170 may utilize the existing components of the imaging system 100, including the compression system 104, x-ray source 122, image receptor 116, etc. In an example, the localization assembly 170 includes clamps, hooks, support brackets, or other attachment means 172 for mounting the assembly 170 to the gantry of the imaging system 100. The clamps or other mounting means 172 may be mated to features of the gantry that support other attached devices, such as a face shield. Examples of localization assemblies are described in U.S. Pat. No. 10,595,954, filed Mar. 2, 2010, which is hereby incorporated by reference in its entirety.

The assembly 170 may include handles 174, which may facilitate transport and/or mounting of the assembly. Attachment elements 172 and a guidance module 175 may be positioned between the handles 174. The guidance module 175 may include components for controlling the movement of the biopsy device 186 (e.g., in an x-direction, y-direction, and/or z-direction relative to the gantry of the imaging system 100). A locking mechanism 185 (e.g., a lever) may secure the assembly 170 to the gantry of the imaging system 100. In an example, the locking mechanism 185 may be manually moved between a locked and unlocked position. In addition to a mechanical coupling of the assembly 170 and the gantry of the imaging system 100, the assembly 170 may also be electrically and communicatively coupled with the imaging system 100, such as via wired connection 187, which may be adapted to connect to a port at the gantry of the imaging system 100. For example, each handle 174 may include one or more electrical connectors which enable communication between a control module 196 and the guidance module 175 such that a medical professional may move the control module to either handle 174 as a matter of preference. The control module 196 includes a user interface that enables a medical professional to control a localization procedure using the assembly 170. The control module 196 includes a display for displaying a status or other information about the localization procedure and one or more user interface elements (e.g., buttons) for controlling movement of one or more elements of the assembly 170 during the localization procedure.

The assembly 170 may also include a fixed support arm 177 that extends from the guidance module 175 to an arm connector 179. In an example, the arm connector 179 couples an angular support arm 178 to the fixed support arm 177 at a fixed angle. Although an arm connector 179 is shown in FIGS. 1B-1D, other adjustment mechanisms for varying an angle of displacement between the angular support arm 178 and the fixed support arm 177 are also appreciated. A holster mount 180 may be moveably coupled to the angular support arm 178. Movement of the holster mount 180 may be linear along the length of the angular support arm 178. Movement of the holster mount 180 along with angular support arm 178 (e.g., substantially in the z-direction) may be mechanically controlled (e.g., via the guidance module and associated motors) and/or may be manually controlled (e.g., via an adjustment element, such as a knob 176). The holster mount 180 is adapted to receive a holster 188 at an attachment mechanism 181. The holster 188 is adapted to receive and secure a localization device 186. The device 186 may include an element insertable into the breast 102. A support 182 may be coupled to the angular support arm 178 and/or the holster mount 180 for stabilization of the device 186. In the depicted example, the device 186 is a biopsy device, such as an Eviva vacuum assisted biopsy device manufactured and sold by Hologic, Inc. As described herein, other devices that are used for other interventional medical procedures are contemplated.

The fixed angle between the angular support arm 178 and the fixed support arm 177 (e.g., as fixed at the arm connector 179) may be an offset that reduces or prevents one or more portions of the assembly 170 from interfering with an x-ray beam generated at the x-ray source 122. In an example, the fixed angle may be 10 degrees, although it is readily appreciated that the offset angle may vary and is largely a matter of design choice and may be varied based on particular geometries of the imaging system 100 and available localization tools. Angling the arm 178 (and by consequence the localization device 186) allows the localization device 186 to be advanced to a desired location within a breast 102 without the localization device or other elements of the assembly 170 introducing artifacts into an x-ray image based on the x-ray beam from the x-ray source 122. In a broadest sense, the assembly 170 allows angling a localization element or other implement to reduce or prevent visual artifacts during x-ray imaging.

FIG. 1D shows a assembly 170, including a device 186, coupled to the imaging system 100. In the example shown in FIG. 1D, the device 186 is a biopsy device 186, including a needle 190. The needle 190 of the biopsy device 186 is guided by the assembly 170 in at least an x-direction and y-direction. The biopsy device 186 may be positioned at x and y coordinates such that movement of the biopsy device 186 in the z-direction (e.g., towards the breast, as may be along an offset angle described above) results in a tip of the needle 190 intersecting the breast 102 at an intersection point. Task lighting 125 may be provided by a light source 123 to illuminate a working area of the imaging system 100, including at least a portion of the surface 194 of the breast 102. The surface 194 of the breast 102 may be exposed at an opening 192 of the foam compressive element 108 compressing the breast 102 via the compression system 104. The task lighting 125 projected from the light source 123 may assist a medical professional during a procedure using the imaging system 100. The light source 123 may be coupled to the assembly 170, such as at an end of the angular support arm 178 and/or the holster mount 180, between the breast and the angular support arm 178 and/or the holster mount 180.

In addition to, or as an alternative to, the light source 123 projecting the task lighting 125, the light source 123 may project a visual indicator 127 onto the surface 194 of the breast 102. The visual indicator 127 may align with the intersection point of the tip of the needle 190 with the surface 194 of the breast 102 along an intersection path (e.g., as the biopsy device 186 is moved in the z-direction, which may be along an offset angle described above). As the assembly 170 and/or doctor positions the biopsy device 186 and the needle 190, the light source 123 may move accordingly, relative to the surface 194 of the breast 102. For example, the light source 123 may be moved in an x-or y-direction relative to the breast 102, the light source 123 may be moved in a z-direction to increase or decrease a distance between the light source 123 and the surface 194 of the breast 102, and/or the offset angle (e.g., angle of the angular support arm 178 and/or the holster mount 180 relative to the z-axis) of the light source 123 may change. Based on the change in position and/or orientation of the light source 123 relative to the surface 194 of the breast 102, the light source 123 may change the projection of the task lighting 125 and/or visual indicator 127. Because the light source 123 is eccentric from the needle 190, at least a change in distance between the light source 123 and the surface 194 of the breast 102 requires re-positioning of at least the visual indicator 127 to counteract a parallax effect and to maintain the projection of the visual indicator 127 at the intersection point of the tip of the needle 190 with the surface 194 of the breast. For example, the light source 123 may adjust the visual indicator 127 to maintain alignment of the visual indicator 127 with the intersection point of the tip of the needle 190 with the surface 194 of the breast 102. In another example, the light source 123 may adjust the task lighting 125 to continue to illuminate at least a portion of the surface 194 of the breast 102.

FIG. 1E depicts, from a viewing plane P in FIG. 1C looking towards the angular support arm 178, a perspective view of the light source 123 of the imaging system 100. The light source 123 may include a light emitter 198, an optical engine 197, and a printed circuit board (PCB) supporting other processing components. Other display components are appreciated. The light source 123 is capable of high-resolution display and advanced light control, such as control of one or more pixels of a display based on a distance between the surface 194 of the breast 102 and the light source 123. Power to the light source 123 may be provided from the imaging system 100 along or inside of the angular support arm 178. The light emitter 198 may include one or more light emitting diodes (LEDs) that are controllable by the optical engine 197 and components of the PCB board 199. In an example, the light source 123 may use digital light processing (DLP), such as the TI DLP®. In another example, the light source 123 is a back-projected liquid crystal display (LCD) with LEDs.

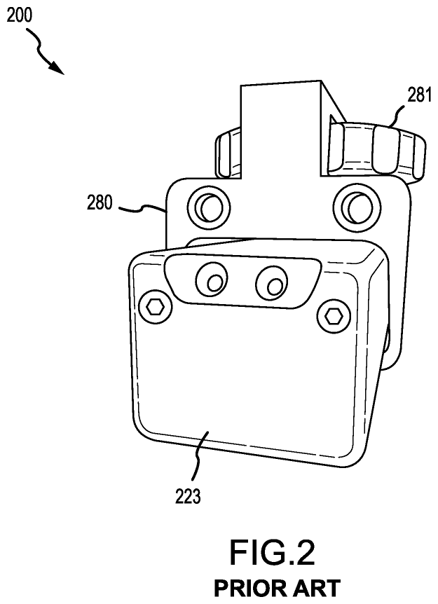

FIG. 2 depicts a prior art configuration of an end of an angular support arm 200 of an imaging system. The angular support arm 200 includes a holster mount 280 secured to the angular support arm 200 via an attachment mechanism 281. Unlike the light source 123 describe above with respect to FIGS. 1A-1E, in the prior art, a light source 223 is capable of providing task lighting, but is not capable of providing any other projections (such as a visual indicator), and is not capable of adjusting, modifying, re-positioning, or otherwise changing any light projected from the light source 223. In the prior art, the light source 223 may be one or more light emitting diodes (LEDs).

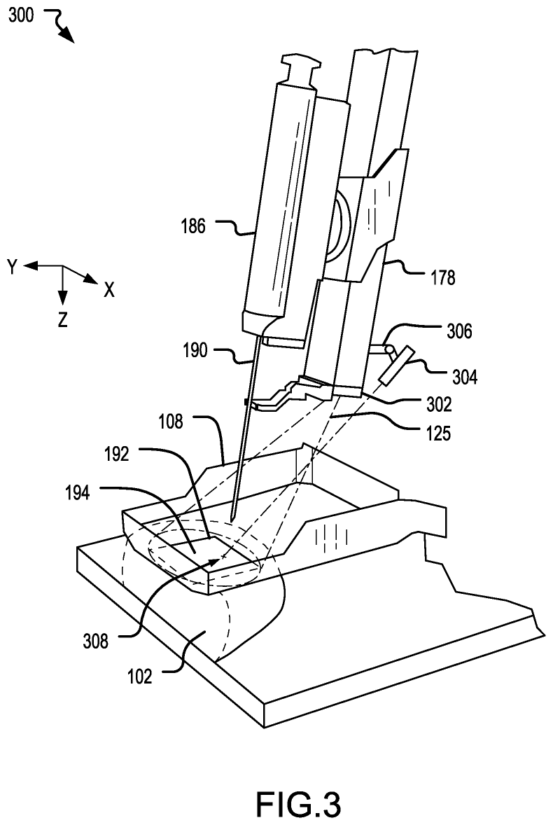

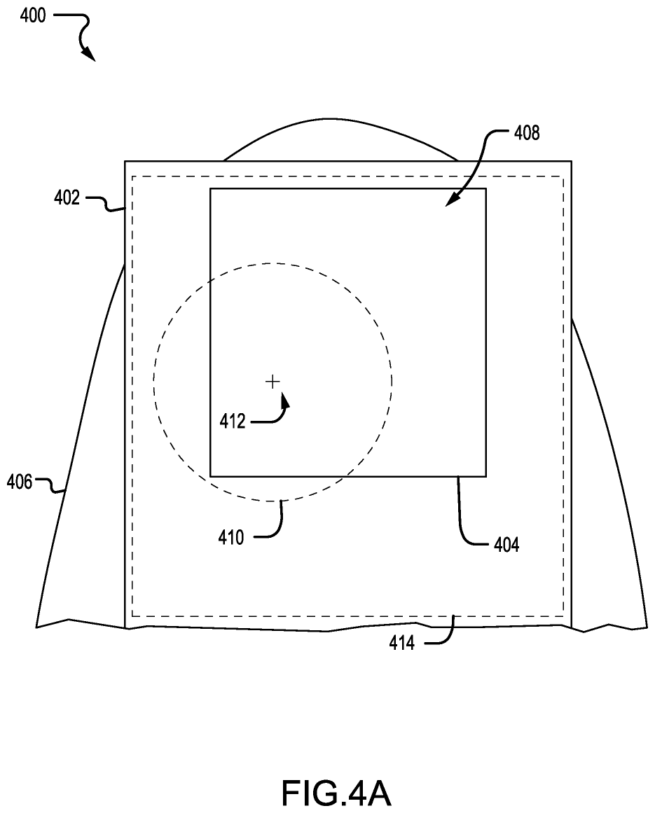

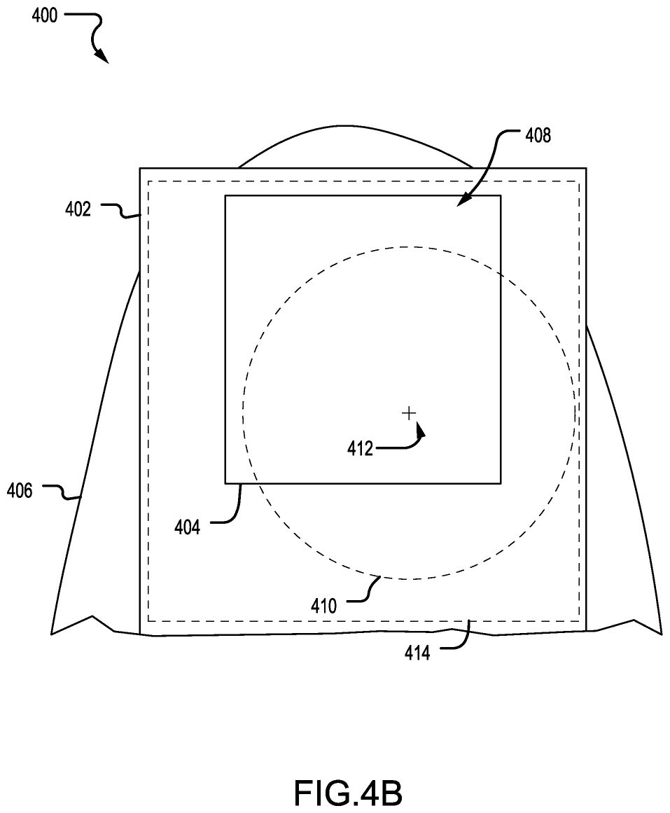

FIG. 3 depicts an imaging system 300 with two light sources 302, 304, instead of a single light source (such as the light source 123 shown in FIG. 1D). As shown in FIG. 3, a first light source 302 is capable of projecting task lighting 125 and a second light source 304 is capable of projecting a visual indicator 308. Visual characteristics of the task lighting 125 from the first light source 302 may or may not be controllable (e.g., as described with respect to FIGS. 1D-1E and FIGS. 4A-4B). In an example, the first light source 302 may be LEDs.

The projection of the visual indicator 308 from the second light source 304 is controllable. For example, the position of the visual indicator 308 on the surface 194 of the breast 102 may be adjusted based on a micro-electromechanical system 306 (MEMS 306). As shown in FIG. 3, the MEMS 306 may angle, position, or otherwise move the second light source 304 relative to the surface of the breast 102 and the localization device 186. For example, as further described herein, the second light source 304 may be adjusted by the MEMS 306 to counteract a parallax effect caused by movement of the second light source 304 toward or away from the surface 194 of the breast 102. As an alternative to a MEMS 306 that moves the second light source 304, the MEMS 306 may instead angle, position, or otherwise move a mirror between the second light source 304 and the surface 194 of the breast 102 to adjust a light path of the projection of the visual indicator 308. The second light source 304 may be coupled to the imaging system in a position that is cleanable, unobstructed, and unlikely to be contacted. In an example, the second light source 304 is a laser.

FIGS. 4A-4B depict top-down views of a breast 406 compressed by a compression device 402 illuminated by task lighting 410 and a visual indicator 412. In FIGS. 4A-4B, the task lighting 410 and the visual indicator 412 are variable relative to the surface 408 of the breast 406, as controlled by one or more light sources described herein (e.g., light sources 123, 302, 304). In FIG. 4A, the visual indicator 412 aligns with a first target incision point (or first intersection point) and in FIG. 4B, the visual indicator 412 aligns with a second target incision point (or second intersection point). As shown, the compression device 402 includes an opening 404 exposing a surface 408 of the breast 406. The breast 406 is positioned such that the visual indicator 412 is projected inside of the opening 404 on the surface 408 of the breast 406 (e.g., to allow interaction with the breast tissue at the position of the visual indicator 412).

Characteristics of the task lighting 410 may be controllable by the light source(s). For example, the size and brightness of the task lighting may be based on user preference. A brightness or intensity of the light may be controlled based on color, such as pure white, a shade of grey, a shade of yellow, a shade of red, a shade a blue, etc. The brightness or intensity may be adjusted relative to an ambient light in a room in which the imaging system is located, such that the task lighting is brighter or more intense than the ambient light in the room. In an example, the brightness or intensity of the task lighting may be adjusted automatically based on the ambient light in the room, as may be detected by a light detector. The size of the task lighting 410 may include all or a portion of the surface 408 of the breast 406 exposed by the opening 404 of the compression device 402. Additionally or alternatively, the task lighting may illuminate all or a portion of a projection area 414 of the light source(s). The projection area is the area capable of being illuminated by a light source. In an example where multiple light sources are projecting light onto a breast 406 (e.g., light sources 302, 304 in FIG. 3), the projection area of each light source may be the same or different. The position of the task lighting 410 may be controllable within the projection area 414. For example, as shown in FIGS. 4A-4B, the task lighting 410 may be centered around a visual indicator 412 projected on the surface 408 the breast 406 with a projection area 414. Alternatively, the task lighting 410 may be centered on the surface 408 of the breast 406 exposed by the opening 404, regardless of the position of the visual indicator 412.

The visual indicator 412 is visually distinguishable from the task lighting 410. For example, the visual indicator 412 may be a different color than the task lighting 410 or different brightness or intensity than the task lighting 410. For instance, the visual indicator may be green or red. The visual indicator 412 may be a variety of shapes, such as a crosshair, a dot, an oval, a rectangle, a line, etc. Additionally, the visual indicator 412 may be any size. In an example, a largest dimension of the visual indicator 412 is less or equal to 3 inches, 2 inches, or one inch. In another example, the longest dimension of the visual indicator 412 is between 0.25 inches and 2 inches.

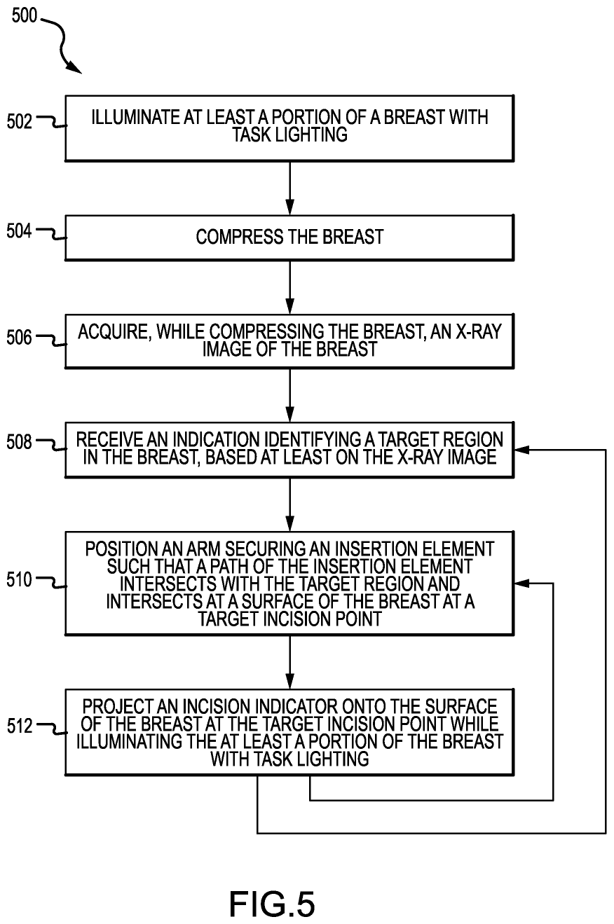

FIG. 5 depicts an example method 500 for projection onto a breast of a patient. The method 500 begins at operation 502, where at least a portion of a breast is illuminated with task lighting. The task lighting may be projected by one or more light source(s) described herein. Characteristics of the task lighting may be controllable, such as brightness or intensity, color, size, shape, etc. For example, the task lighting may be projected using DLP.

At operation 504, the breast is compressed. At operation 506, while compressing the breast, an x-ray image of the breast is acquired. The breast may be compressed with a compression system, such as immobilizer unit 104, to secure the breast during imaging and/or targeting procedures. One or more x-ray images of the breast are acquired with components of the imaging system described above. The x-ray images may be referenced to determine one or more target regions in the breast, based on abnormalities identified via the one or more x-ray images. The target regions are identified in three-dimensional space in the breast.

At operation 508, an indication identifying a target region in the breast is received. The indication may be based at least on the x-ray image acquired at operation 506. For example, identification of the target regions in the x-ray image(s) allows the imaging system to determine a position of the target region(s) in three-dimensional space (e.g., x, y, and z coordinates).

At operation 510, an arm securing an insertion element is positioned. The arm is positioned such that a path of the insertion element intersects with the target region identified at operation 508 and intersects at a surface of the breast at a target incision point. The position of the arm may be known or otherwise detected by the imaging system. The imaging system may mechanically move the arm in an x-direction and a y-direction, relative to the compressed breast. Movement of the arm in the z-direction (e.g., as may be along on an offset angle) may be controllable by a user of the imaging system.

At operation 512, an incision indicator (e.g., visual indicator 127, 310) is projected onto the surface of the breast at the target incision point. The incision indicator may be projected concurrently with illuminating the at least a portion of the breast with task lighting, described at operation 502. The incision indicator may be projected from a light source that is the same or different than the light source projecting the task lighting. For example, the task lighting and the incision indicator may be projected from a single light source (e.g., light source 123 shown in FIG. 1D). In another example, the task lighting may be projected from a first light source and the incision indicator may be projected from a second light source (e.g., light sources 302, 304 shown in FIG. 3). The light source of the incision indicator is capable of adjusting the projection of the incision indicator, based on a position of the arm. In an example where the light source of the incision indicator is DLP, the DLP may perform mathematical operations to adjust the position of the incision indicator.

Additionally, the method 500 may include terminating projection of the incision indicator. The incision indicator may be independently controllable from the task lighting. For example, projection of the incision indicator may be terminated without terminating projection of task lighting. For instance, projection of the task lighting may be terminated subsequently to termination of projection of the incision indicator. The incision indicator may be terminated after a user nicks the breast tissue at the target incision point. Termination of the projection of the incision indicator may include receiving an indication to review projection of the incision indicator. For example, a user may indicate, at the imaging system, to review characteristics of the incision indicator (e.g., brightness, shape, color, size, on/off, etc.). The user may then indicate to turn off the incision indicator.

Operations 508-512 may repeat as required or desired. For example, the at least one target region may include a first target region and a second target region in the breast, associated with different intersection points at the surface of the breast. In an example, the indication received at first operation 508 identifies the first target region and the indication received at repeated operation 508 identifies the second target region. At repeated operation 510, the arm securing the insertion element is re-positioned such that the path of the insertion element intersects with the surface of the breast at the second target region and a second target incision point (which may be different than the target incision point of the first target region). As described above, the position of the arm securing the insertion element may be known or otherwise detected by the imaging system. At repeated operation 512, the incision indicator is adjusted (e.g., based on the re-positioning of the arm) and projected onto the surface of the breast at the second target incision point. Although the above discussion of repeated operations 508-512 describes projection of one incision indicator at a time, it is appreciated that multiple incision indicators (e.g., for multiple target regions) may be displayed concurrently. In this instance, the incision indicators may have different visual characteristics to indicate which target region is currently targeted based on the position of the arm.

Additionally or alternatively, operations 510-512 may repeat as required or desired. As further described herein, a position of the arm may be adjustable in an x-direction, y-direction, and/or z-direction. As the arm is adjusted in any direction (e.g., in the z-direction along an offset angle to move the insertion element closer to the target region in the breast), the projection of the incision indicator is adjusted to project the incision indicator at the target incision point. The position of the projected incision indicator may be automatically adjusted based on a change in a distance between the surface of the breast and a projector projecting the incision indicator. For example, the position of the projected incision indicator may be adjusted to counteract a parallax effect. Additionally or alternatively, projection of the incision indicator may be automatic based on detecting that the arm is positioned such that the path of the insertion element intersects with the breast at the at least one target region.

The method 500 may assist a user of an imaging system to accurately and precisely nick, or create an incision in, a surface of a breast (e.g., at the projected incision point). The nick in the breast tissue at the surface of the breast may ease insertion of the insertion element into the breast for marking and/or biopsying of the target region in the breast.

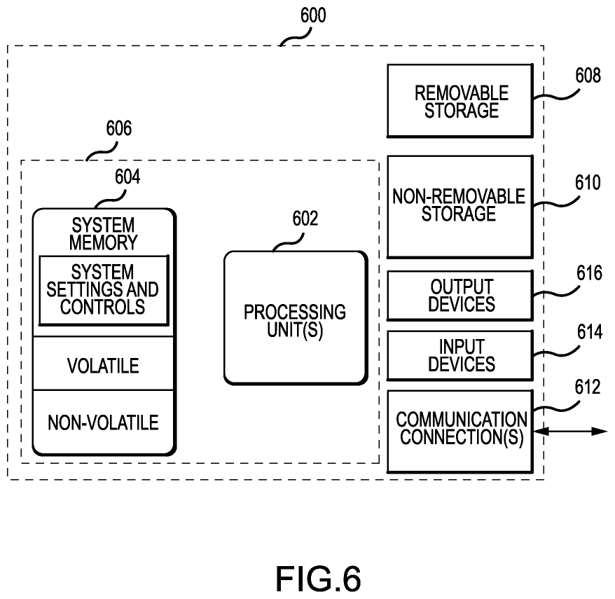

FIG. 6 illustrates an example suitable operating environment 600 for projection onto a breast of a patient, as described herein. In its most basic configuration, operating environment 600 typically includes at least one processing unit (or processor) 6602 and memory 604. Depending on the exact configuration and type of computing device, memory 604 (storing, instructions to perform projection of an image onto a specimen) may be volatile (such as RAM), non-volatile (such as RAM, flash memory, etc.), or some combination of the two. This most basic configuration is illustrated in FIG. 6 by dashed line 606. Further, environment 600 may also include storage devices (removable, 608, and/or non-removable, 610) including, but not limited to, magnetic or optical disks or tape. Similarly, environment 600 may also have input device(s) 614 such as keyboard, mouse, pen, voice input, etc. and/or output device(s) 616 such as a display, speakers, printer, etc. Also included in the environment may be one or more communication connections 612, such as LAN, WAN, point to point, etc. In embodiments, the connections may be operable to facility point-to-point communications, connection-oriented communications, connectionless communications, etc.

Operating environment 600 typically includes at least some form of computer readable media. Computer readable media can be any available media that can be accessed by one or more processing units (or processors) 602 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory medium which can be used to store the desired information. Computer storage media does not include communication media.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, microwave, and other wireless media. Combinations of the any of the above should also be included within the scope of computer readable media.

The operating environment 600 may be a single computer operating in a networked environment using logical connections to one or more remote computers. The remote computer may be a personal computer, a server, a router, a network PC, a peer device or other common network node, and typically includes many or all of the elements described above as well as others not so mentioned. As an example, the operating environment 600 may be shared between one or more imaging systems, such as imaging system 100. The logical connections may include any method supported by available communications media. Such networking environments are commonplace in offices, enterprise-wide computer networks, intranets, and the Internet.

### EXAMPLES

Illustrative examples of the systems and methods described herein are provided below. An embodiment of the system or method described herein may include any one or more, and any combination of, the clauses described below:

Clause 1. A method for projecting an incision indicator onto a breast of a patient, the method comprising: compressing the breast; acquiring, while compressing the breast, an x-ray image of the breast; receiving an indication identifying at least one target region in the breast, based at least on the x-ray image; detecting that an arm securing an interventional element is positioned such that a path of the interventional element intersects with the breast at the at least one target region and intersects at a surface of the breast at a target incision point; and projecting an incision indicator onto the surface of the breast at the target incision point while illuminating the at least a portion of the breast with task lighting.

Clause 2. The method of clause 1, the method further comprising: adjusting the projected incision indicator based on a change in a distance between the surface of the breast and a projector projecting the incision indicator.

Clause 3. The method of clause 2, the method further comprising: adjusting the projected incision indicator to counteract a parallax effect.

Clause 4. The method of any of clauses 1-3, wherein the at least one target region includes a first target region and a second target region, the method further comprising: receiving a selection of the first target region, wherein the incision indicator is projected based on the path of the interventional element intersecting the first target region; receiving a selection of the second target region; detecting that the arm is repositioned such that the path of the interventional element intersects with the second target region; and adjusting the projection of the incision indicator to intersect the path of the interventional element at the second target region on the surface of the breast.

Clause 5. The method of any of clauses 1-4, wherein projecting the incision indicator is automatic based on detecting that the arm is positioned such that the path of the insertion element intersects with the breast at the at least one target region.

Clause 6. The method of any of clauses 1-5, the method further comprising: receiving an indication to review projection of the incision indicator; and terminating projection of the incision indicator.

Clause 7. The method of clause 6, wherein the task lighting is illuminated subsequent to terminating projection of the incision indicator.

Clause 8. The method of any of clauses 1-7, wherein the incision indicator is at least one of: a crosshair; a dot; an oval; a rectangle; and a line.

Clause 9. The method of clause 8, wherein the incision indicator comprises a selectable color.

Clause 10. An apparatus for projecting an incision indicator onto a breast of a patient, the apparatus comprising: an x-ray source capable of selectively moving relative to the breast; an x-ray detector; a compression system for compressing the breast, the compression system disposed between the x-ray source and the x-ray detector; an arm for securing an interventional element; a task lighting source disposed between the x-ray source and the compression system; an indicator source disposed between the x-ray source and the compression system, wherein the indicator source is coupled to the arm; a processor; and memory storing instructions that, when executed by the processor, cause the apparatus to perform a set of operations comprising: illuminating at least a portion of the breast with the task lighting source; detecting that the arm is in a position such that a path of the interventional element intersects with a surface of the breast at a target incision point; and projecting an incision indicator from the indicator source onto the surface of the breast at the target incision point.

Clause 11. The system of clause 10, wherein the tasking lighting source and the indicator source are the same.

Clause 12. The system of any of clauses 10-11, wherein the tasking lighting source and the indicator source are a projector.

Clause 13. The system of clause 12, the set of operations further comprising: determining a location of the incision indicator in a projection image of the projector, based on a distance between the projector and the surface of the breast.

Clause 14. The system of clause 13, wherein the distance between the projector and the surface of the breast is based on the position of the arm.

Clause 15. The system of any of clauses 10-14, wherein the indicator source is calibrated based on a calibration position of the arm.

Clause 16. The system of any of clauses 10-15, wherein the indicator source is a micro electrical mechanical system (MEMS) including a mirror that is adjustable to control reflection of a laser beam to project the incision indicator onto the surface of a breast.

Clause 17. The system of clause 16, wherein the mirror of the MEMS is adjusted based on a distance between the indicator source and the surface of the breast.

Clause 18. The system of any of clauses 10-17, wherein the indicator source is positioned at an end of the arm.

Clause 19. The system of any of clauses 10-18, wherein the interventional element is a needle or a wire.

Clause 20. An apparatus for projecting an incision indicator onto a breast of a patient, the apparatus comprising: an x-ray source capable of selectively moving relative to the breast; an x-ray detector; a compression system for compressing the breast, the compression system disposed between the x-ray source and the x-ray detector; an arm for securing an interventional element, the arm disposed between the x-ray source and the compression system; and a projector coupled to an end of the arm, the projector capable of selectively projecting task lighting and an incision indicator onto a surface of the breast based at least on a position of the arm and a target incision point.

Although aspects of the present disclosure are described with respect to image analysis of living breast tissue, it should be appreciated that the present disclosure may also be useful in variety of other applications where identifying different densities of cells may improve image analysis, such as imaging excised breast tissue, other tissue, bone, living organisms, body parts, or any other object, living or dead.

As should be appreciated, while the above methods have been described in a particular order, no such order is inherently necessary for each operation identified in the methods. For instance, the operations identified in the methods may be performed concurrently with other operations or in different orders. In addition, the methods described above may be performed by the systems described herein. For example, a system may have at least one processor and memory storing instructions that, when executed by the at least one processor, cause the system to perform the methods described herein.

The embodiments described herein may be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices may be employed to perform the functionality disclosed herein without departing from the scope of the disclosure.

This disclosure describes some embodiments of the present technology with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art. Further, as used herein and in the claims, the phrase “at least one of element A, element B, or element C” is intended to convey any of: element A, element B, element C, elements A and B, elements A and C, elements B and C, and elements A, B, and C.

Although specific embodiments are described herein, the scope of the technology is not limited to those specific embodiments. One skilled in the art will recognize other embodiments or improvements that are within the scope and spirit of the present technology. Therefore, the specific structure, acts, or media are disclosed only as illustrative embodiments. The scope of the technology is defined by the following claims and any equivalents therein.

1. A method for projecting an incision indicator onto a breast of a patient, the method comprising:
compressing the breast;
acquiring, while compressing the breast, an x-ray image of the breast;
receiving an indication identifying at least one target region in the breast, based at least on the x-ray image;
detecting that an arm securing an interventional element is positioned such that a path of the interventional element intersects with the breast at the at least one target region and intersects at a surface of the breast at a target incision point; and
projecting an incision indicator onto the surface of the breast at the target incision point while illuminating the at least a portion of the breast with task lighting.

2. The method of claim 1, the method further comprising:
adjusting the projected incision indicator based on a change in a distance between the surface of the breast and a projector projecting the incision indicator.

3. The method of claim 2, the method further comprising:
adjusting the projected incision indicator to counteract a parallax effect.

4. The method of claim 1, wherein the at least one target region includes a first target region and a second target region, the method further comprising:
receiving a selection of the first target region, wherein the incision indicator is projected based on the path of the interventional element intersecting the first target region;
receiving a selection of the second target region;
detecting that the arm is repositioned such that the path of the interventional element intersects with the second target region; and
adjusting the projection of the incision indicator to intersect the path of the interventional element at the second target region on the surface of the breast.

5. The method of claim 1, wherein projecting the incision indicator is automatic based on detecting that the arm is positioned such that the path of the insertion element intersects with the breast at the at least one target region.

6. The method of claim 1, the method further comprising:
receiving an indication to review projection of the incision indicator; and
terminating projection of the incision indicator.

7. The method of claim 6, wherein the task lighting is illuminated subsequent to terminating projection of the incision indicator.

8. The method of claim 1, wherein the incision indicator is at least one of:
a crosshair;
a dot;
an oval;
a rectangle; and
a line.

9. The method of claim 8, wherein the incision indicator comprises a selectable color.

10. An apparatus for projecting an incision indicator onto a breast of a patient, the apparatus comprising:
an x-ray source capable of selectively moving relative to the breast;
an x-ray detector;
a compression system for compressing the breast, the compression system disposed between the x-ray source and the x-ray detector;
an arm for securing an interventional element;
a task lighting source disposed between the x-ray source and the compression system;
an indicator source disposed between the x-ray source and the compression system, wherein the indicator source is coupled to the arm;
a processor; and
memory storing instructions that, when executed by the processor, cause the apparatus to perform a set of operations comprising:
illuminating at least a portion of the breast with the task lighting source;
detecting that the arm is in a position such that a path of the interventional element intersects with a surface of the breast at a target incision point; and
projecting an incision indicator from the indicator source onto the surface of the breast at the target incision point.

11. The system of claim 10, wherein the tasking lighting source and the indicator source are the same.

12. The system of claim 10, wherein the tasking lighting source and the indicator source are a projector.

13. The system of claim 12, the set of operations further comprising:
determining a location of the incision indicator in a projection image of the projector, based on a distance between the projector and the surface of the breast.

14. The system of claim 13, wherein the distance between the projector and the surface of the breast is based on the position of the arm.

15. The system of claim 10, wherein the indicator source is calibrated based on a calibration position of the arm.

16. The system of claim 10, wherein the indicator source is a micro electrical mechanical system (MEMS) including a mirror that is adjustable to control reflection of a laser beam to project the incision indicator onto the surface of a breast.

17. The system of claim 16, wherein the mirror of the MEMS is adjusted based on a distance between the indicator source and the surface of the breast.

18. The system of claim 10, wherein the indicator source is positioned at an end of the arm.

19. The system of claim 10, wherein the interventional element is a needle or a wire.

20. An apparatus for projecting an incision indicator onto a breast of a patient, the apparatus comprising:
an x-ray source capable of selectively moving relative to the breast;
an x-ray detector;
a compression system for compressing the breast, the compression system disposed between the x-ray source and the x-ray detector;
an arm for securing an interventional element, the arm disposed between the x-ray source and the compression system; and
a projector coupled to an end of the arm, the projector capable of selectively projecting task lighting and an incision indicator onto a surface of the breast based at least on a position of the arm and a target incision point.
